(19) United States
(12) Patent Application Publication
(10) Pub. No.: US 2011/0046544 A1
(43) Pub. Date: Feb. 24, 2011

(54) APPARATUS AND METHOD FOR RELEASING A MASS FROM A BIOLOGICAL CAVITY

(75) Inventor: Antony Hugh Beeley, Siesta Park (AU)

Correspondence Address: ANDREWS KURTH LLP, 1350 I STREET, N.W., SUITE 1100, WASHINGTON, DC, 20005, US

(73) Assignee: ANTONY HUGH BEELEY, Siesta Park (AU)

(21) Appl. No.: 12/990,274

(22) PCT Filed: Apr. 30, 2009
(86) PCT No.: PCT/AU2009/000538

§ 371 Date: Oct. 29, 2010

(30) Foreign Application Priority Data
May 1, 2008 (AU) 2008902138

(51) Int. Cl. A61M 37/00

(52) U.S. Cl. 604/23

(57) ABSTRACT

An apparatus (1) for releasing a mass from a wall portion of a cavity, the apparatus (1) comprising at least one gas providing member (5) and at least one support means (4) adapted to in use deploy the at least one gas providing member (5) to the cavity to provide gas thereto to release the mass from the wall portion of the cavity.

### FIELD OF THE INVENTION

The present invention relates to the medical field and more particularly to apparatus and methods applied during surgical procedures for releasing and/or removing masses from biological cavities.

### BACKGROUND ART

Surgical procedures related to the extraction from biological cavities of large space-occupying masses, (such as an unborn child, organ or tumour, are widespread. In numerous circumstances it is necessary to extract, for example, a tumour. Also, in some cases organ transplants are performed which imply the release and extraction of an organ from a biological cavity. Similarly, the Caesarean section (C-section) is a form of childbirth in which a surgical incision is made through a female abdomen and uterus to deliver one or more babies. In all cases it is required that the integrity of the mass to be removed is maintained. Therefore, particular care has to be taken when extracting the large space-occupying masses.

In many circumstances difficulties arising during the extraction of large space-occupying masses from biological cavities that contain them are due to suction arising from a vacuum that develops between the bodies to be extracted and the inner walls of the cavities on attempted extraction. For example, in some cases during the Caesarean section difficulties can arise in overcoming suction to release of the baby's head from the uterine inner walls, cervix and vagina. In some instances this may require an assistant to push up the baby's head via the vagina, although such a procedure can result in the introduction of infections. Even in some circumstances the baby's head is pulled forcibly out of the mother's pelvis the resulting vacuum possibly injuring the baby's ears, eyes or respiratory tract. Similar problems may be encountered during the extraction of tumours or organs. In these cases it can be important to maintain the integrity of the organs or tumours for future transplants and/or biopsies. Accordingly, there is a need to develop methods and apparatus for the safe release and extraction of a mass such as baby, organ or tumour contained in a biological cavity.

### SUMMARY OF THE INVENTION

According to a first aspect of the invention there is provided an apparatus for releasing a mass from a wall portion of a cavity, the apparatus comprising at least one gas providing member and at least one support means adapted to in use deploy the at least one gas providing member to the cavity to provide gas thereto to release the mass from the wall portion of the cavity.

Preferably, the gas providing member comprises a gas flow path having a distal portion for communication with the cavity and a proximal portion for communication with a source of gas.

Preferably, the source of gas is the atmosphere.

Preferably, the gas flow path is integrally formed with the support means.

Preferably, the gas flow path comprises at least one conduit.

Alternatively, the gas flow path comprises at least one groove.

In an arrangement the support means comprise a surgical device.

In a further arrangement the support means comprise means adapted to be worn or otherwise fitted on at least one finger.

Preferably, the gas flow path comprises at least one tissue drain.

Preferably, the means adapted to be worn on at least one finger comprises a finger stall.

In yet a further arrangement the support means comprise means adapted to be worn on a hand.

Preferably, the means adapted to be worn on a hand comprise a glove.

Preferably, the glove is a fingerless glove.

According to a second aspect of the invention there is provided a glove for releasing a mass from a wall portion of a cavity comprising a proximal opening for receiving a hand of a user and a gas flow path extending to a distal portion of the glove for delivering gas to the wall portion of the cavity to release the mass.

In an arrangement, the glove is a fingerless glove.

Preferably, the glove comprises a plurality of portions for receiving fingers of a user, the plurality of portions comprising the gas flow path.

Preferably, the gas flow path extends along a dorsal portion of the glove.

Preferably, the gas flow path comprises at least one conduit.

Alternatively, the gas flow path comprises at least one groove.

According to a third aspect of the invention there is provided a finger stall for releasing a mass from a wall portion of a cavity comprising a proximal opening to receive a finger of a user and a gas flow path extending to a distal portion of the finger stall portion for delivering gas to the wall portion of the cavity to release the mass.

Preferably, the gas flow path comprises at least one tissue drain.

According to a fifth aspect of the invention there is provided a surgical device for releasing a mass from a wall portion of a cavity comprising a proximal end adapted to deliver a distal portion of the surgical device to the cavity and a gas flow path extending to the distal portion for delivering gas to the wall portion of the cavity to release the mass.

According to a sixth aspect of the invention there is provided a method to free at least one mass from at least one wall portion of a cavity comprising the steps of:
deploying at least one gas providing member to the cavity;
providing a gas via the at least one gas providing member to the at least one wall portion of the cavity; and
releasing the at least one mass from the at least one wall portion.

Preferably, the mass is a foetus.

According to a seventh aspect of the invention there is provided a method to extract a mass from a cavity comprising the steps of:
deploying at least one gas providing member to the cavity;
providing a gas via the at least one gas providing member to the at least one wall portion of the cavity;
releasing the at least one mass from the at least one wall portion; and
extracting the at least one mass from the cavity.

Preferably, the mass is a foetus.

### BRIEF DESCRIPTION OF THE DRAWINGS

The present invention will be better understood by reference to the following description of one specific embodiment thereof as shown in the accompanying drawings, in which:

FIG. 1 is a perspective view of an apparatus according to a first embodiment of the invention configured as a surgical glove;

FIG. 2 is a top view of the apparatus according to the first embodiment;

FIG. 3a is a top view of the apparatus according to the second embodiment;

FIG. 3b is an alternative arrangement of the apparatus according to the second embodiment;

FIG. 4 is a face view of apparatus according to a third embodiment of the invention configured as a finger stall;

FIG. 5 is a side view of the apparatus of FIG. 4;

FIG. 6 is a top view of the apparatus according to a fourth embodiment of the invention.

FIG. 7 is a cross section taken thought the plane 6-6 of FIG. 6; and

### DETAILED DESCRIPTION OF SPECIFIC EMBODIMENT(S)

In a surgical operation which the aim is to extract a large space-occupying mass from a body cavity (such as a large tumour from the abdominal cavity or a baby's head out of the pelvis in a Caesarean section with the baby in the common head down position), the hand of the person performing the operation can be passed around the mass but its extraction is made difficult by the inability of the air to pass into the potential space behind or below the mass in which the operator's hand is lying. This creates a severe vacuum effect on attempting to extract the mass which resists removal of the mass and may cause injury to the baby's ear, eyes or respiratory tract in the case of Caesarean section. The aim of the invention is to provide a passage for air to enter this potential space thereby abolishing the vacuum effect and thus allowing easy delivery of the mass out of the body cavity or the baby's head out of the pelvis and avoiding any risk of vacuum injury. The vacuum effect is more marked the lower the baby's head is lying in the pelvis of the mother if the Caesarean section is an emergency procedure which becomes necessary after labour is established.

The embodiments shown in the drawings relate to surgical procedures involving Caesarean sections during which difficulties can arise in overcoming the vacuum effect to release of the baby's head from the uterine inner wall, cervix, and vagina.

The embodiments are directed to apparatus and methods for releasing the vacuum that develops deep in the pelvis in order to facilitate extraction of the baby.

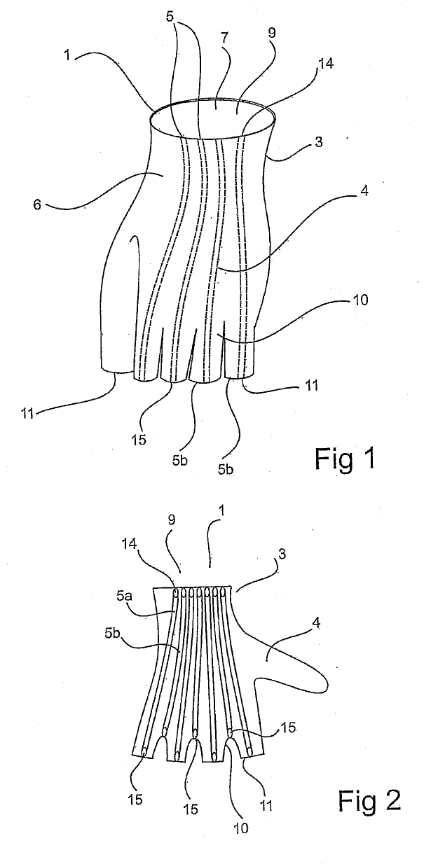

Referring to FIGS. 1 and 2, the apparatus 1 according to the first embodiment comprises a support means 3 configured as a glove 4 adapted to be fitted onto one hand (not shown) of a user (being the surgeon or another person involved in the birth of the baby), and a plurality of conduits 5 for delivering gas in the form of air to the zone at which the vacuum exists.

The conduits 5 are attached to or formed integrally with the glove 4. With this arrangement, the user is able to deploy the conduits 5 to the location where the unborn baby is in contact with the inner surface of the uterine wall thus providing air for breaking the vacuum and freeing the head of the baby from the inner surface of the uterine wall cervix and vagina deep in the pelvis.

The glove comprises a proximal opening 9 to receive the surgeon's hand and distal openings 11 to receive the surgeon's fingers. In the illustrated arrangement there are seven conduits of which four conduits 5a correspond to the distal openings 11 and the remaining three conduits 5b correspond to the junctions 10 between the distal opening. Conduits 5a extend from the proximal opening 9 to the distal openings 11. Each conduit 5 has an inlet end defining an inlet orifice 14 and an outlet end defining an outlet orifice 15. The orifices 15 may be bevelled atraumatic orifices in order to avoid injuries to the foetus.

In the arrangement illustrated the conduits 5 are located adjacent the dorsal portion of the glove 4. In another arrangement the conduits may be located elsewhere, such as for example the palmar or lateral portions of the glove 4.

Glove 4 is configured as a fingerless glove which can be worn over a surgical glove that covers the entire surgeon's hand (not shown). Glove 4 may be worn if needed during the extraction procedure and removed when not needed anymore.

Glove 4 may be made of any appropriate material such as latex, vinyl or nitrile rubber or mixtures of them. The conduits 5 may be any type of suitable conduits such as, for example tissue drains.

In operation, the user presents his or her hand fitted with glove 4 to the zone where the head of the unborn child is in contact with the inner surface of the uterine wall. The conduits 5 establish flow paths between the inlet orifices 14 which communicate with atmospheric air and the outlet orifices 15 which communicate with the vacuum zone. This delivers air to the region adjacent the inner surface of the uterus inner wall for breaking the vacuum and freeing the head of the baby from the inner surface of the uterine wall. Once the head is released the infant may be extracted.

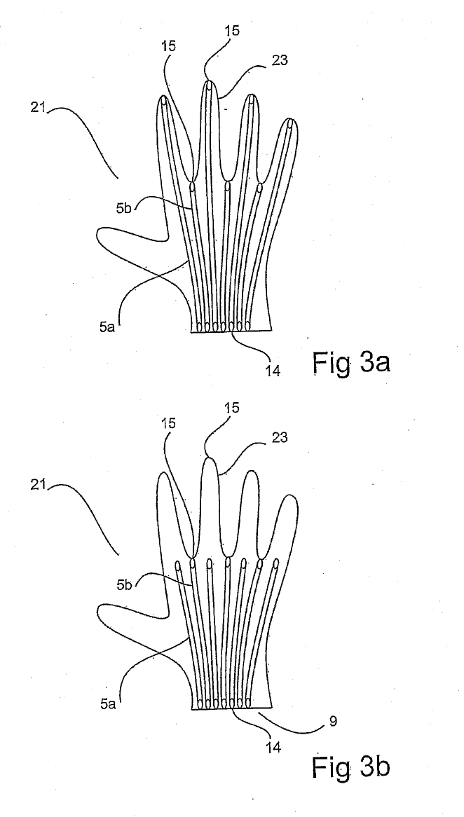

FIG. 3 shows an apparatus according to a second embodiment of the invention. The apparatus according to the second embodiment is similar to the apparatus according to the apparatus of the first embodiment and similar reference numerals are used to identify similar parts. In this second embodiment the glove 21 is configured as a conventional glove in the sense of having portions 23 into which the fingers of the users are fully received. Portions 23 comprise ends 12 which cover the user's fingertips. The conduits 5a extend from the proximal opening 9 to ends 12 so as to located orifices 15 at the user's fingertips. Alternatively, orifices 15 may be located at any location of the glove 21. For example, as shown FIG. 3b, conduits 5a has the same length than conduits 5b locating thus orifices 15 at the user's knuckles. The outlet orifice 15 may be configured as bevelled atraumatic orifices in order to avoid injuries to the unborn baby.

Glove 21 may be used by surgeons in only one hand. The other hand can be covered by a normal surgical glove.

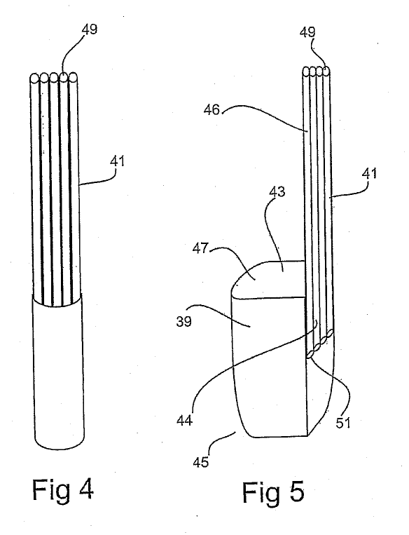

Referring to FIGS. 4 and 5, the apparatus according to a third embodiment of the invention comprises a support means 37 configured as a finger stall 39 adapted to be fitted onto one to two or more fingers (not shown) of a user (being surgeon or other person involved in the birth of the baby). The finger stall comprises at least one conduit 41 for delivering gas in the form of air to the zone at which the vacuum exists.

The conduits 41 are attached to or formed integrally with the finger stall 39. With this arrangement, the user is able to deploy the conduits 41 to the location where the head of the unborn baby (not shown) is in contact with the inner surface of the uterine wall thus providing air for breaking the vacuum and freeing the head from the inner surface of the uterine wall (not shown).

The finger stall 39 according to the third embodiment of the invention (see FIG. 4) comprises a proximal opening 43 to receive a finger (not shown) and a distal portion 45 to cover the fingertip (not shown). The fingerstall 39 may be worn by the user by introducing the finger into proximal opening 43 and covering the fingertip with the distal portion 45.

As shown in FIGS. 4 and 5, the conduits 41 comprise a distal portion 44 and a proximal portion 46. Distal portion 44 comprising outlet orifices 51 is attached to the finger stall 39 so that outlet orifices 51 are located at a position between the proximal opening 43 and the distal portion 45 of the fingerstall. Alternatively, the conduits 41 may extend all the way to the distal end 45. The outlet orifice 51 may be configured as bevelled atraumatic orifices in order to avoid injuries to the unborn baby. Proximal portion 46, comprising inlet orifices 49, is located at a position with respect to the proximal opening 43 of the finger stall 39 such that inlet orifices 49 communicate with atmospheric air during the procedure of releasing the unborn baby from the uterine wall. This allows for conduits 41 to establish flow paths between the inlet orifices 49 which communicate with atmospheric air and the outlet orifices 51 which communicate with the vacuum zone. Thus, air can be delivered to the region adjacent the inner surface of the uterine inner wall for breaking the vacuum and freeing the head of the baby from the inner surface of the uterine wall. The finger stalls 39 may be used by surgeons in one or more fingers. The fingers may be covered by normal surgical gloves.

The finger stall 39 may comprise latex, vinyl or nitrile rubber or mixtures of them. Conduits 41 may be any type of suitable conduits or tissue drains.

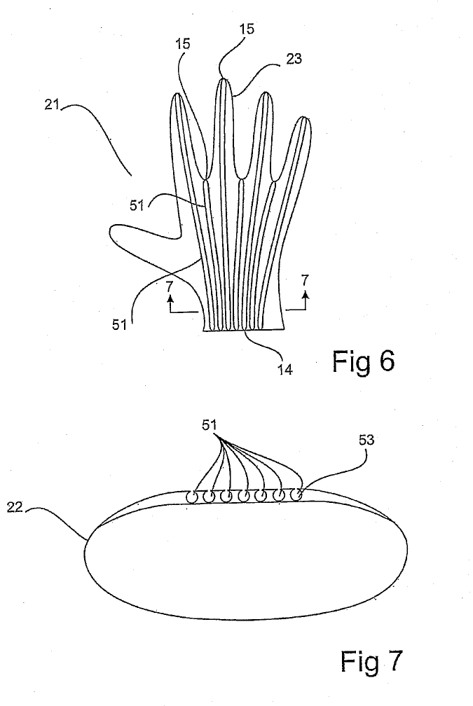

FIGS. 6 to 7 show an apparatus according to a fourth embodiment of the invention. The apparatus according to the fourth embodiment is similar to the apparatus according to the apparatus of the second embodiment and similar reference numerals are used to identify similar parts. In this fourth embodiment the gas providing members are grooves 51 formed in the material of the glove 21. As shown in FIGS. 6 and 7 grooves 51 comprise open channels 53 extending from the proximal opening 9 to distal openings 11.

A fifth embodiment of the invention, which is not shown, the support means is configured as a surgical device which carries one or more of the conduits The surgical device may be of any appropriate type, such as for example, an organ extraction device.

Modifications and variations as would be apparent to a skilled addressee are deemed to be within the scope of the present invention.

Further, it should be appreciated that the scope of the invention is not limited to the scope of the embodiments disclosed. By way of example, the apparatus and method according to the invention may be suitable to release and extract organs and tumours, as well as organisms (such as a foetus) as described in relation to the embodiments.

Throughout the specification and claims, unless the context requires otherwise, the word “comprise” or variations such as “comprises” or “comprising”, will be understood to imply the inclusion of a stated integer or group of integers but not the exclusion of any other integer or group of integers.

1. An apparatus for releasing a mass from a wall portion of a cavity, the apparatus comprising:
at least one gas providing member; and
at least one support means adapted to in use deploy the at least one gas providing member to at least one location of contact of the mass and the wall portion to provide gas thereto to diminish or eliminate vacuum formed between the mass and the wall portion to assist release of the mass from the wall portion of the cavity.

2. An apparatus according to claim 1 wherein the gas providing member comprises a gas flow path having a distal portion for communication with the at least one location of contact of the mass and the wall portion and a proximal portion for communication with a source of gas.

3. An apparatus according to claim 2 wherein the source of gas is the atmosphere.

4. An apparatus according to claim 2 wherein the gas flow path is integrally formed with the support means.

5. An apparatus according to claim 2 wherein the gas flow path comprises at least one conduit.

6. An apparatus according to claim 2 wherein the gas flow path comprises at least one groove.

7. An apparatus according to claim 1 wherein the support means comprise a surgical device.

8. An apparatus according to claim 1 wherein the support means comprise means adapted to be worn or otherwise fitted on at least one finger.

9. An apparatus according to claim 8 wherein the gas flow path comprises at least one tissue drain.

10. An apparatus according to claim 8 wherein the means adapted to be worn on at least one finger comprises a finger stall.

11. An apparatus according to claim 1 wherein the support means comprise means adapted to be worn on a hand.

12. An apparatus according to claim 11 wherein the means adapted to be worn on a hand comprise a glove.

13. An apparatus according to claim 12 wherein the glove is a fingerless glove.

14. A glove for releasing a mass from a wall portion of a cavity comprising:
a proximal opening for receiving a hand of a user; and
a gas flow path extending to a distal portion of the glove for delivering gas to at least one location of contact of the mass and the wall portion to diminish or eliminate vacuum formed between the mass and the wall portion to assist release of the mass.

15. A glove according to claim 14 wherein the glove is a fingerless glove.

16. A glove according to claim 14 wherein the glove comprises a plurality of portions for receiving a fingers of user, the plurality of portions comprising the gas flow path.

17. A glove according to claim 14 wherein the gas flow path extends along a dorsal portion of the glove.

18. A glove according to claim 14 wherein the gas flow path comprises at least one conduit.

19. A glove according to claim 14 wherein the gas flow path comprises at least one groove.

20-22. (canceled)

23. A method to free at least one mass from at least one wall portion of a cavity comprising the steps of:
deploying at least one gas providing member to at least one location of contact of the at least one mass and the at least one wall portion;
providing a gas via the at least one gas providing member to the at least one location of contact of the at least one mass and the at least one wall portion to diminish or eliminate a vacuum formed between the mass and the wall portion; and
releasing the at least one mass from the at least one wall portion.

24. A method according to claim 23 wherein the mass is a foetus.

25-30. (canceled)

31. A method according to claim 20 further comprising the steps of extracting the at least one mass from the cavity.
